(19) United States
(12) Patent Application Publication
(10) Pub. No.: US 2003/0069510 A1
(43) Pub. Date: Apr. 10, 2003

(54) Disposable vital signs monitor

(76) Inventor: Herbert J. Semler, Portland, OR (US)

Correspondence Address: MARGER JOHNSON & MCCOLLOM PC, 1030 SW MORRISON STREET, PORTLAND, OR, 97205, US

(21) Appl. No.: 09/971,507

(22) Filed: Oct. 4, 2001

(51) Int. Cl. A61B 5/0428

(52) U.S. Cl. 600/509

(57) ABSTRACT

The invented cardiac monitor is in a flexible, nominally flat planar form having integral gel electrodes, a sticky-back rear surface, an internal flex circuit capable of sensing, recording and playing out several minutes of the most recently acquired ECG waveform data and a front surface that includes an outplay port preferably having one or more snap connectors compatible with a lead harness from an n-lead recorder. The monitor has a relatively short battery life, as it is intended for limited-term use and, after a single recording and outplay session it may be disposed of, e.g. discarded or recycled. Preferably, the monitor uses one or more zinc-air batteries the air inlet ports of which may be selectively configured, as by folding or otherwise manipulating the monitor's expanse, to either activate or deactivate particular recording or outplay modes of operation of the monitor. The circuitry within the flex circuit inner layer of the monitor's expanse may preferably be implemented by very large scale integration (VLSI) techniques by use of a custom integrated circuit (IC) that performs any necessary sensing, recording and outplay functions. The circuitry may be digital, and may include an analogue-to-digital (A/D) converter, a microprocessor with associated memory and a digital-to-analogue (D/A) converter. Alternatively, the circuitry may take the form of a direct analogue storage device between the electrodes and an outplay port that is compatible with existing or custom output and hard-copy devices. In a suggested alternative embodiment, the monitor further may be remotely controlled by telemetry to deliver pacer or defibrillation pulses to the patient.

### BACKGROUND OF THE INVENTION

The present invention relates generally to vital signs monitors whereby a patient's electrocardiograph (ECG), for example, is sensed and graphically recorded, e.g. as waveform data. More particularly, it concerns a thin flat, flexible monitor having integral electrodes that is extremely lightweight and may be adhered to the patient's chest during a recording session and that may be removed for local or remote outplay, as by mailing it to a physician's or diagnostician's lab for playout, diagnostic and/or archival purposes and ultimate disposal. The invented vital signs monitor lends itself to other continuous graphic waveform e.g. electroencephalograph (EEG) or pulse oximetry, or static, e. g. pulse-rate, blood pressure, glucose level, blood-oxygen level, vital signs monitoring, as well as telemetric control as for delivering pacer or defibrillation pulses to the monitored patient.

Some cardiac monitors having integral electrodes have been worn around the wrist, as described in U.S. Pat. No. 5,289,824 entitled WRIST-WORN ECG MONITOR, which issued Mar. 1, 1994. The high functional density of such cardiac monitors, and the provision therein of trans-telephonic communication of ECG waveform data to a remote physician site, render such monitors extremely useful in our increasingly busy and mobile society. More recent advances have rendered such high functionality and lightweight portability in the form of a credit card-shaped and -sized monitor such as the known HEARTCARD™ monitor. Such a product requires manual placement and slight pressure by the user on the monitor against the chest with the integral dry electrodes in contact with the skin and the manual depression of a record button. Such a product also requires the placement of a telephone call to a physician's office and the careful playing out of recorded, digitized, frequency-shift keyed (FSK) ECG waveform data via a telephone's mouthpiece. The HEARTCARD™ monitor is intended for long-term use, and thus is enclosed in a durable rigid housing, is provided with long-life batteries, and is supplied with a carrying case.

### SUMMARY OF THE INVENTION

Briefly, the invented cardiac monitor is in a flexible, nominally flat planar form having integral gel electrodes, a sticky-back rear surface, an internal flex circuit capable of sensing, recording and playing out several minutes of the most recently acquired ECG waveform data and a front surface that includes an outplay port preferably having one or more snap connectors compatible with a lead harness from an n-lead recorder. The monitor has a relatively short battery life, as it is intended for limited-term use. After the patient has completed a recording session, the monitor may be simply sent in the mail to the prescribing physician for diagnostic and archival purposes. The physician or technician may play out the recorded ECG waveform data by activating an outplay mode of operation, and the patient's cardiography may be studied. The tiny, inexpensive monitor may then be disposed of, e.g., discarded or recycled. In a suggested alternative embodiment, the monitor further may be remotely controlled by telemetry to deliver pacer or defibrillation pulses to the patient.

Preferably, the monitor uses one or more zinc-air batteries the air inlet ports of which may be selectively configured, as by folding or otherwise manipulating the monitor's expanse, to either activate or deactivate particular recording or outplay modes of operation of the monitor. Thus, recording may be accomplished by simply opening the monitor, which activates the zinc-air batteries, and pasting the monitor on the patient's chest. When a recording session is complete, e.g. when a cardiac event has been detected or upon the initiative of the patient who may have sensed such an event, the monitor may be folded again thus deactivating the recorder by removing battery power therefrom. At the physician site, the opening again of the monitor may automatically activate an outplay mode of operation in which a connected n-lead recorder presents a strip chart recording of the patient's cardiography.

The circuitry within the flex circuit inner layer of the monitor's expanse may preferably be implemented by very large scale integration (VLSI) techniques by use of a custom integrated circuit (IC) that performs any necessary sensing, recording and outplay functions. The circuitry may be digital, and may include an analogue-to-digital (A/D) converter, a microprocessor with associated memory and a digital-to-analogue (D/A) converter. Alternatively, the circuitry may take the form of a direct analogue storage device having a differential amplifier front-end for sensing the amplitude of the analogue ECG input and having constant gain between input and output, the latter of which is coupled operatively with the outplay port. Thus, outplay may be analogue or digital in form, and may be infrared (IR), audio (trans-telephonic), or electrical, e. g. an RS-232 serial input/output (I/O) port compatible with a connected personal computer (PC) or a lead-set compatible with an n-lead, e. g. a 12-lead, strip chart recorder. Other suitable recording and outplay means may be used such as a printer, tape, disk, CD-ROM, TV, VCR LCD, etc.

These and additional objects and advantages of the present invention will be more readily understood after consideration of the drawings and the detailed description of the preferred embodiment which follows.

### BRIEF DESCRIPTION OF THE DRAWINGS

FIG. 1 is a lateral, cross-sectional view of the cardiac monitor adhered to a cardiac patient's chest, showing some of the detail of its interior construction.

FIG. 2 is a schematic circuit diagram of the cardiac monitor made in accordance with a preferred embodiment of the invention.

FIG. 3 is a schematic circuit diagram of the cardiac monitor made in accordance with an alternative embodiment of the invention.

FIG. 4 is an isometric view of the monitor in a flat configuration in which it is useful for recording, and illustrates the laminar structure of the monitor of its preferred embodiment.

FIG. 5 is an isometric view of the monitor in a folded configuration that, in accordance with one aspect of the invention, protects its integral electrodes, powers-down its circuitry, saves its battery and readies it for a recording or outplay session.

FIG. 6 is an enlarged cross-sectional edge view of the invented apparatus taken generally along the lines 6-6 in FIG. 4.

FIG. 7 is an enlarged, fragmentary cross-sectional view of the apparatus taken generally along the lines 7-7 in FIG. 4.

### DETAILED DESCRIPTION

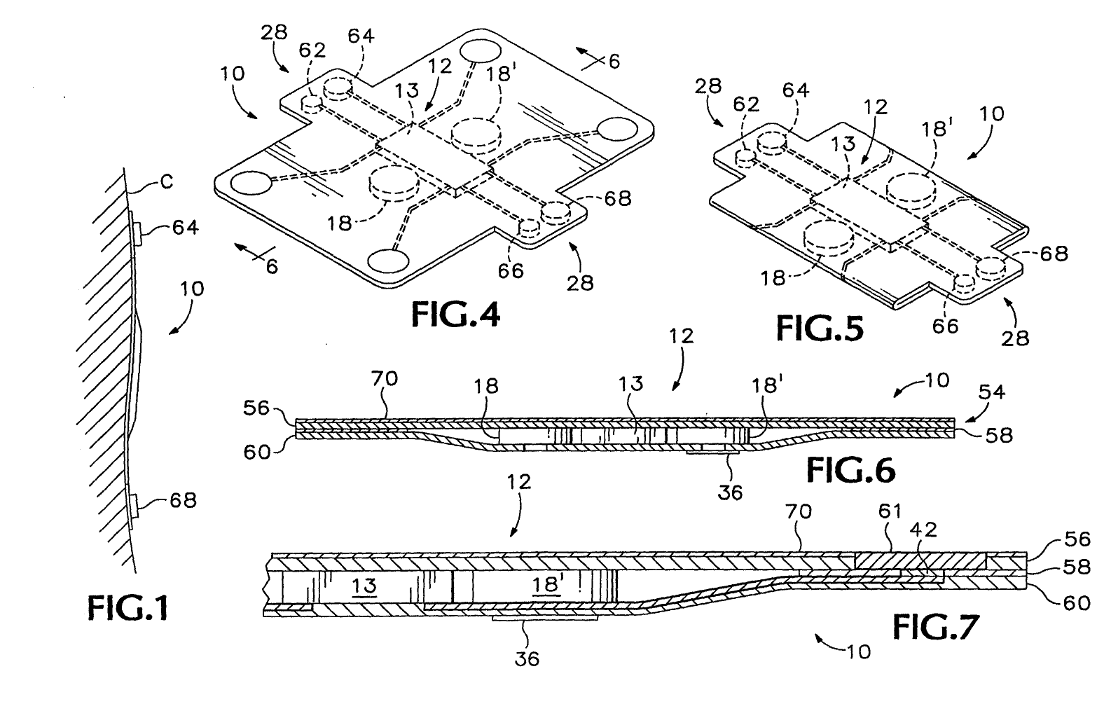

Referring first to FIG. 1, the invented disposable vital signal, e.g. cardiac, monitor is indicated generally at 10 adhered to the chest C of a medical patient. It will be appreciated that, because monitor 10 is integral, self-contained and adherent, the patient is free to move about performing everyday tasks without concern for lead-sets or external connections or manipulation of the monitor or any operator controls thereon. Because of its tiny size and weight, and because of its flexibility, the invented monitor resembles a medium-sized adhesive bandage, and thus provides for extremely convenient, affordable, comfortable and accurate vital signs monitoring and recording for children or men and women of all sizes and builds.

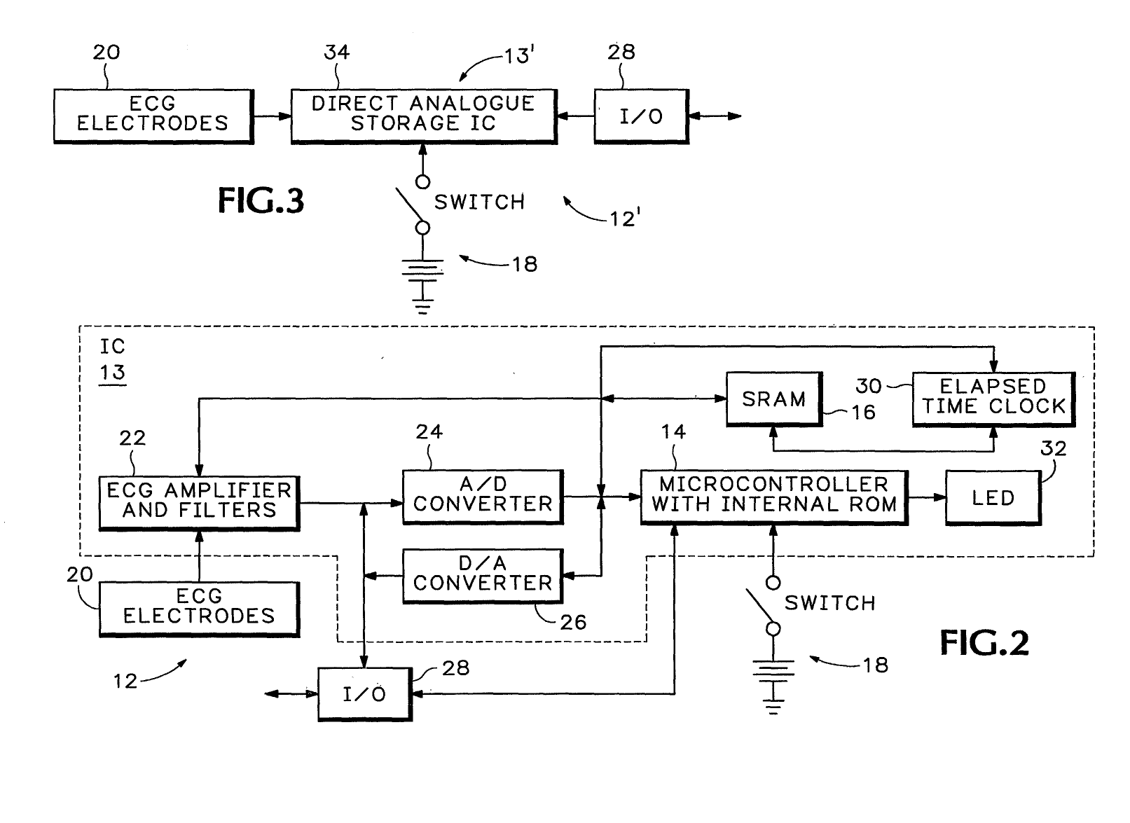

Monitor 10 will be understood to be capable easily and quickly of being removed by the patient at the end of a monitoring and recording session, thereby enabling waveform data recorded therein to be outplayed. Those skilled in the art will appreciate that outplaying may be via or to a local or remote presentation device such as a printer, tape, disk, CD-ROM, TV, VCR, LCD, etc. An outplay port is provided in monitor 10, as will be described in more detail by reference to FIGS. 2 and 3, in any of a variety of forms preferably including a set of snap connectors that are plug-compatible with the installed base of 12-lead strip-chart recorders found in diagnostic clinics around the world.

Those of skill in the art will appreciate that monitor 10 alternatively may utilize the world-wide web, or Internet, as a conduit or destination for the vital signs data stored therein. Thus, a so-called Bluetooth or other wireless, e.g. infrared or radio frequency (RF), interface port may be provided—compatible with the small size, thinness and flexibility of monitor 10—and vital signs data may be telecommunicated to nearby or remote sites via the Internet for playback, viewing, analysis, recording, archiving, etc. So-called Instant Messaging, a common feature of e-mail, could be used to post cardiograms to a receiving or diagnostic clinic or individual cardiologist situated anywhere in the world from a cardiac patient also situated anywhere in the world. Indeed, Instant Messaging could be used for duplex communications between a patient and a physician, however remote from one another, of vital signs data and other message content.

Thus, in accordance with the preferred embodiment of the invention and method for its use monitor 10 may be purchased over-the-counter by a medical patient and upon completion of a recording session may be delivered, as by mail or walk-in or drive-through, to a diagnostic clinic for outplay, oversight, diagnostics and archival recording. Because it is meant for limited-term use, and is extremely inexpensive to manufacture, after its recorded data is outplayed at the clinic, monitor 10 may be disposed of, e.g. discarded or recycled, much like a disposable flash camera. Of course, those of skill in the art will appreciate that, within the spirit and scope of the invention, monitor 10 instead may be reused, as by recharging or replacing one or more batteries, which it is appreciated typically might require some rebuilding of the novel laminar structure and thus may not be cost effective.

The invented vital signs monitor, then, may be seen most broadly to include a flexible generally planar expanse that includes a front surface and a rear surface including a region capable of being adhered to a patient's skin, with the rear surface bearing two or more, e.g. four, electrodes. Preferably, the monitor includes also an outplay port, as will be seen, that may take the form of a general-purpose input/output (I/O) port that is wired or wireless and that enables an interior flexible circuit sandwiched between the rear and front surfaces of the expanse to communicate either unidirectionally or bidirectionally with an external device such as a remote transmitter/receiver or processor or simple hardcopy device.

Those of skill in the art will appreciate that FIGS. 1, 4 and 5 show monitor 10 in a given size that may be suitable for adherence to the chest of a person of average size. Within the spirit and scope of the invention, disposable vital signs monitor 10 may assume a variety of sizes, e.g. adult (e.g. over eighteen years), youth (e.g. between 11 and eighteen) and child (e.g. under eleven) sizes, compatible with more individualized torsos. Such may be particularly beneficial for monitoring sudden infant death syndrome (SIDS) most likely to strike a pre-adolescent child. Importantly, the thin, lightweight, flexible monitor imposes little or no burden or inconvenience even for a person having the most fragile frame or tiny body. Thus, SIDS among other anomalies or syndromes may be monitored, and lives may be saved, using the invented disposable vital signs monitor even in the case of a preemie of extremely low birth weight and size, and the same or other vital signs may be monitored even in the case of a weak and/or disabled elder.

High-risk athletes or non-athletes also are candidates for use of the invented vital signs monitor. Athletes could wear the monitor under their normal athletic attire during a sporting event, without adverse effect on their performance, but with the possibility of discovering and treating an anomaly. High-risk patients, for example, during the post-myocardial infarction (MI) or post-coronary angioplasty (PCTA) phases of their treatment may be equipped with the vital signs monitor to record and early detect or diagnose any anomalous vital signs that are monitored thereby during critical post-operative or post-treatment phases of their lives. Those of skill in the art also will appreciate that the invented vital signs monitor may be used on non-human patients. In other words, veterinarians might use the vital signs monitor on dogs, cats, horses or other animals in the delivery of veterinary health care.

Turning now to FIG. 2, a schematic diagram of the interior flexible circuit or circuitry of the preferred embodiment of the invention is shown at 12. It will be appreciated that circuitry 12 preferably is implemented in one or more integrated circuits or other integral components of extremely light weight, low profile and small footprint. Such may be one or more highly integrated circuits (IC), as is taught by the above-referenced patent disclosure. Those of skill in the art will appreciate that circuitry 12 may provide more or less functionality than is described herein in terms of a preferred embodiment of the invention, within the spirit and scope of the invention. For example, circuitry 12 may include pulse generation means that, via the same gel electrodes as those used for monitoring, deliver a series of low-wattage pacer pulses or a high-wattage defibrillation pulse to the patient's heart.

Referring now in more detail to FIG. 2, it may be seen that circuitry 12 preferably includes a micro-controller 14, or a microprocessor having internal read-only memory (ROM) suitably programmed; non-volatile, e. g. static, read-and-write memory (SRAM) 16 for variable and vital signs waveform data recording or storage; at least one battery 18 selectively operable to power and thus enable the circuit to perform its sensing, recording, producing and playing functions. Battery 18 preferably is of the air seal type, e.g. one or more zinc-air batteries of which only one is shown in FIG. 2, having an integral SWITCH for selectively applying power to the remainder of circuitry 12; plural electrodes such as the preferred gel-type ECG electrodes indicated generally at 20; signal-sensing circuitry such as ECG amplifiers and filters 22 operatively connected with electrodes 20; an analogue-to-digital converter (ADC) 24 that operatively couples the electrodes to the digital processor operatively coupled, in turn to the memory; a digital-to-analogue converter (DAC) operatively coupled with the digital processor and the memory and operatively coupled, in turn to an outplay port; and an input and/or output (I/O) or more simply an outplay port indicated generally at 28 for conveying sensed and recorded vital signs waveform data to a remote outplay or recording device for medical diagnostic purposes and, optionally, for receiving command or control data from a nearby preferably wireless transmitter for cardiac pacing or defibrillating purposes.

Those skilled in the art will appreciate that, by logical extension, disposable vital signs monitor 10 may be of the so-called Holter monitor-type characterized as providing multiple-lead cardiac monitoring. Such a monitor might use any suitable arrangement or number of leads both within the perimeter of the monitor's body, as illustrated in FIGS. 1, 4 and 5, or having external leads attached to thin, lightweight cables extending therefrom. In such an arrangement, the monitor itself yet might be disposable after, say, 24-48 hours worth of cardiac data are monitored and continuously recorded. Alternatively, a looping memory scheme may be used, as is known but as will be described briefly below, to selectively record only more pertinent, suspected event, data for much longer periods of time, say 1-2 months. Those of skill in the art will appreciate that the volume of data recordable in memory, whether continuously or selectively, increases step-wise periodically, as semiconductor memory densities increase and prices decrease.

It will be appreciated that such circuitry 12 as described above readily may be integrated into one or more custom integrated circuits (ICs) that takes up little space, whether in the plane of monitor 12 or normal thereto. Preferably, one IC 13 is used to reduce cost and flex circuit and interconnect complexity, as suggested by the simple configuration of monitor illustrated in FIG. 4, to be described below.

Those skilled in the art will appreciate that circuitry 12 also may include an elapsed time clock 30 for data-and-time stamping of recorded vital signs waveform data and one or more audio or visual annunciators such as beepers or light-emitting diodes (LEDs), e. g. LED 32, for indicating to the patient or clinician the status of monitor 10, i.e. whether it contains recorded vital signs waveform data that is ready for outplay.

Within the spirit and scope of the invention, circuitry 12 may provide other useful functions. For example, a scrolling or looping memory function may be provided by which SRAM 16 is partitioned into one or more looping buffers for the capture-store of a predetermined time duration of data, with the most recently sensed, i.e. the latest recorded, data always present therein and with the least recently sensed, i.e. the oldest recorded, data lost. In this way, circuitry 12 equipped to trigger on a detected cardiac anomaly may halt recording of data into the looping memory thereby to capture for outplay a cardiac data window that is pertinent to, because it is time proximate to, the triggering cardiac event. Numerous alternative or additional functions may be provided by circuitry 12, within the spirit and scope of the invention, as it is understood that functionality readily may be added by reprogramming or masking a state or logic controller such as microcontroller 14.

FIG. 3 schematically illustrates an alternative embodiment of the circuitry that may be used within monitor 10 to implement the basic sensing, recording and outplaying functions. Circuitry 12′ provides such functions in the form of an analogue signal recorder such as those used to customize greeting cards by permitting the sender to record a message which is outplayed automatically when the recipient opens the greeting card. Such analogue 10 memories, or direct analogue storage devices, such as that indicated at 34 (also designated 13′ to indicate that it is counterpart to digital IC 12 of FIG. 2) are inexpensive to manufacture, and have a recording capacity-because of the unique nature of vital signs waveform data-of recording at least approximately one minute of continuous ECG waveform data sensed by the electrodes, preferably at least approximately two minutes thereof and most preferably at least approximately four minutes thereof.

The differences between the human voice and vital signs graphic waveform data lead to this eight-fold recording capacity increase. The human voice may be reasonably well reproduced by digitizing it at a sampling rate of approximately 4000 Hertz (Hz), whereas accurate cardiac graphic waveform data need be sampled only at approximately 400-500 Hz in order to faithfully reproduce it for a clinician to diagnose the shortest duration arrhythmic, ischemic or other cardiac anomaly. Moreover, because of the analogue nature of the stored data, representing essentially in a single sample the amplitude of a patient's skin potential between two electrodes is possible with direct analogue storage, whereas eight binary bits typically are used to represent a digital representation of such amplitude. Thus, by lowering the sampling rate of such a device, its capacity to record vital signs graphic waveform data is greatly increased to a meaningful level.

Whether monitor 10 stores a digital or an analogue representation of the sensed vital signs waveform signal, it is preferably in accordance with the invention that at least approximately one minute of such sensed vital signs, e.g. ECG, signal be recorded within memory 18 or 18′. More preferably, at least approximately two minutes of such sensed vital signs signal is recorded, and most preferably approximately four minutes of capacity within memory 18, 18′ is provided, thereby rendering monitor 10 useful for multiple event or medium-term monitoring of patient vital signs. It will be appreciated that the useful capacity of memory 18 or 18′ may be effectively increased by the use of scrolling or looping memory and automatic trigger event-detection such that the greatest fraction of recorded vital signs signal is useful in representing the patient's vital signs for overview and analysis by a diagnostician.

Other modifications are required to such a direct analogue storage device to render it suitable for vital signs monitoring. First, the input amplifier section must be made differential so match the differential input from the electrodes, as may be readily accomplished by those of skill. Second, the gain of the device must be made substantially constant, or of substantially consistent unity gain, from such differential input to output. Such straightforwardly may be accomplished by simply disabling the automatic gain control (AGC) of the conventional direct analogue storage device.

Operatively connected to the differential input terminals of such analogue storage device 34 is an electrode pair, or ECG electrodes 20 made in accordance with the preferred embodiment of the invention, which electrodes of course carry a differential signal representing the patient's skin potential (typically a third and fourth electrode provide a common baseline for the differential pair). Operatively connected to the output buffer electronics of such analogue storage device 34 is bidirectional I/O, or unidirectional outplay, port 28 also made in accordance with the preferred embodiment of the invention, which outplay port of course may take any of the variety of forms described or illustrated herein. One or more identical batteries such as illustrated battery 18 may be used, connected to the analogue storage device preferably via a battery-integral SWITCH, as shown.

As indicated, it is preferable that a reserve battery (not shown in FIGS. 2 and 3 for the sake of clarity, but shown in FIGS. 4, 6 and 7 described below) be provided as back up to primary battery 18 in both the preferred and alternative embodiments illustrated in FIGS. 2 and 3, in case the primary battery fails. Those of skill in the art will appreciate that the primary and reserve batteries may be connected in parallel so that whichever one has sufficient power and has its integral switch (air-powered) will supply the remainder of circuitry 12 or 12′ within monitor 10. Alternatively or additionally, and within the spirit and scope of the invention, one or more larger capacity batteries may be provided, thereby enabling pulse generation circuitry within monitor 10 to deliver relatively high-voltage pacer or defibrillation pulses to the patient.

FIG. 4 shows monitor 10 in a bottom isometric view in its flat configuration for medical patient waveform data recording, i.e. in what will be referred to herein as its second, deployed configuration. In its preferred embodiment, the laminar structure may be seen to take the form of a thin preferably rectangular, generally planar expanse that will be understood by its structure to be flexible. The thin rectangular expanse may be approximately credit card-shaped and sized, or approximately 6.0 cm×9.0 cm×0.4 cm (2.4″×3.6″×0.16″). Those of skill in the art will appreciate: that monitor 10 may take alternative shapes and sizes, within the spirit and scope of the invention. It will also be appreciated that, if made to be credit card-shaped and -sized, monitor 10 may have the additional feature of a ROM magnetic strip on one edge thereof that may be initially programmed to identify the patient to whom the monitor is provided and that may later be read by a suitable magnetic strip reader. Such a ‘smart’ card approach is within the spirit and scope of the invention.

Within a preferably central interior region of monitor 10 are one or more batteries such as primary and reserve zinc-air batteries 18, 18′ operatively interconnected preferably by an air-actuated switch integral therewith to circuitry 12 capable of sensing, recording and outplaying vital signs waveform data such as a patient's ECG waveform. It will be appreciated that primary battery 18 has its air inlet normally exposed on the front surface of the expanse of monitor 10 so that it is operative when monitor 10 in its second, deployed configuration is tightly adhered to the patient's chest as in FIG. 1. Likewise reserve battery 18′, although it may be seen that normally the air inlet of battery 18′ is covered by an air-impermeable sealing tab 36, as shown so that the battery is not normally in operation but may be easily rendered operative by the tab's removal.

Recent advances in battery technologies render far greater performance to disposable vital signs monitor 10. It is believed that a sheet battery is presently under development by the military that could be used to power the relatively low-power requirements of monitor 10 as described herein. Such a battery is made of a special laminar fabric which may be cut to size and which exhibits a sustained electrical potential thereacross capable of powering one or more electrical circuits. Such a recent advance might prove extremely suitable as a suitable alternative to the discrete one or more batteries illustrated herein, because of the similar characteristic flexibility of such sheet batteries and the disclosed monitor, leading to even thinner and more flexible disposable vital signs monitors. One such sheet battery, the Power Paper™ thin battery, is available from Power Paper Ltd., an Israeli corporation. It is contemplated that, within the spirit and scope of the present invention, some or all of the circuitry including the electrodes, the flex circuit, the memory and/or processor chip and the batteries may be integrated into a thin, laminar configuration.

In accordance with a preferred embodiment of the invention, four gel-type electrodes 38, 40, 42, 44 are provided in the four corners of the expanse on the bottom surface thereof for contact with the patient's chest. Preferably, such electrodes which are referred to collectively herein as electrodes 20 are connected with corresponding input terminals of circuitry 12 in accordance with one of the schematic diagrams of FIGS. 2 and 3, discussed above via a flex circuit conductor layer that also connects the batteries with the remaining circuitry. This flex circuit conductor layer is indicated somewhat schematically in FIG. 4 by dashed line pairs extending from circuitry 12 to batteries 18, 18′, to electrodes 20 and to outplay port 28 (this flex circuit is illustrated in more detail in FIG. 7).

It will be appreciated that, alternatively and yet within the spirit and scope of the invention, electrodes 38, 40, 42, 44 maybe of another type of so-called wet electrodes, or even may be dry electrodes as are taught in the above-referenced patent disclosure. It will also be appreciated by those skilled in the art that the number, configuration and spacing of electrodes 20, within the spirit and scope of the invention, may vary depending upon the cardiac (in the case of ECG), cerebral (in the case of EEG) or other vector(s) to be monitored and recorded by monitor 10. It will also be appreciated that electrodes 20 of the gel type are suitable for use in pacer and defibrillation pulse transmission to the patient.

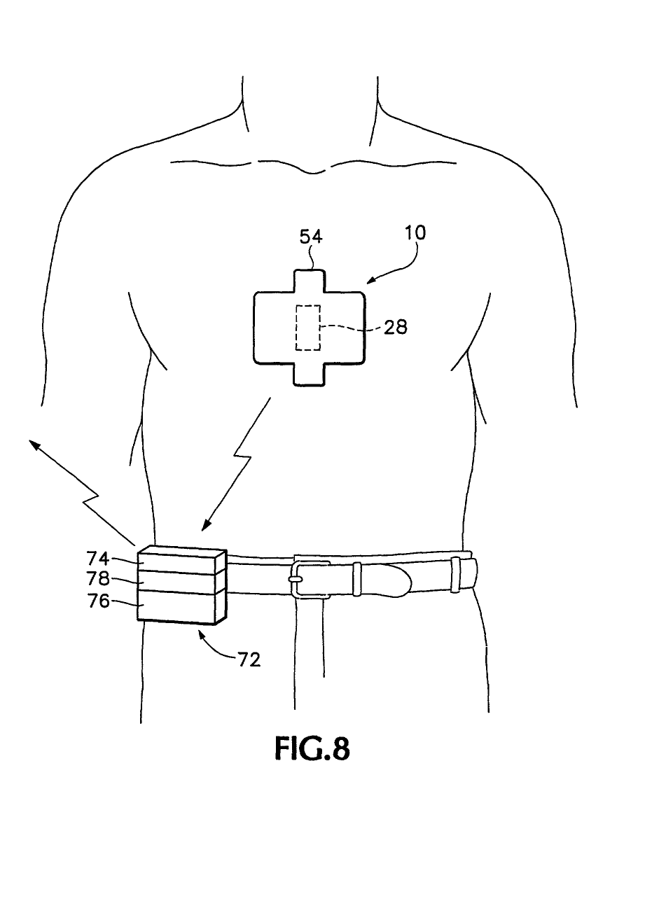

Shown in FIG. 4 as four snap connectors 46, 48, 50, 52 (indicated by dashed lines) and associated I/O routing flex circuitry (in pairs of dashed lines) is I/O or outplay port 28. It will be appreciated that snap connectors 46, 48, 50, 52 may be located anywhere in the flexible expanse of monitor 10 that does not interfere with its use in recording and outplaying sessions. The chosen position permits monitor 10 to be flatly bi-folded as shown in FIG. 5 to seal the air inlets of batteries 18, 18′, while not measurably increasing the overall profile of monitor 10. It will be appreciated that placement of connectors on the rear surface or edge surfaces of monitor 10 may be possible within the spirit and scope of the invention, without interfering with adherence by monitor 10 to the patient's chest or accurate sensing of vital signs thereat, depending upon their physical configuration.

It will also be appreciated that edge connectors may be used that are within the slight overall profile of monitor 10. For example, many so-called PCMCIA modem cards present a phono jack for telephone cord connection in the extremely thin edge regions thereof, and such might be used with a different type of I/O port envisioned by the invention. With wireless communication schemes such as IR or RF or audio (e. g. trans-telephonic), extremely low- or no-profile I/O ports alternatively may be provided. For example, IR may be-used to provide bidirectional wireless communication between the monitor and a nearby receiver, akin to the use of a wireless remote control on a television or a vehicle security system. All are within the spirit and scope of the invention. Alternatively, monitor 10 may be equipped with an internal modem as part of circuitry 12, thereby enabling direct telephone line connections for remote outplay. All such producing and playing of waveform data functions of circuitry within the expanse are contemplated and are within the spirit and scope of the invention.

Brief reference to FIG. 5 shows monitor 10 in what will be referred to herein as its first, stowed configuration in which the air inlet to battery 18 is substantially closed or covered by one folded expanse, thereby rendering battery 18 inoperable, via its integral SWITCH, to supply power to circuitry 12. In this stowed configuration, monitor safely and confidently may be transported or stored e.g. in an SW flat envelope-without decreasing battery life and without risking loss of any patient vital signs waveform data stored in its non-volatile memory. It will be appreciated that a paper backing sheet cut approximately to the rectangular shape and size of monitor 10 when flat might be placed on the adhesive-coated rear surface thereof when monitor 10 is not being used to record vital signs data thereby protecting electrodes 20 from wear or contamination and a patient's or clinician's hands from stickiness. Those of skill in the art will appreciate that manipulation of the monitor's expanse from the first, stowed configuration shown in FIG. 5 to the second, deployed configuration shown in FIG. 4 selectively operates the battery (e. g. by supplying its air inlet with air by unblocking it), thereby to power and thus enable the circuit to operate, e. g. for recording or outplay.

It will be appreciated that, in accordance with an alternative embodiment of the invention, monitor 10 need not be folded or configured specially for stowage. In such an alternative embodiment, the air inlet of battery 18 might be sealed by simply placing a sealing tab thereover, i.e. to save primary battery 18 when it is not needed just as reserve battery 18 is saved when it is not needed. Such a flat configuration of monitor 10 whether in operation or not lends itself to the ‘smart’ card magnetic encoding described above. Nevertheless, by the use of air seal batteries in a disposable vital signs monitor, no physical pushbutton switch or other operator control is required to operate monitor 10 in all of its intended functional roles. Thus, unnecessary cost, weight and complexity in monitor 10 are avoided.

As may be seen by reference to FIGS. 6 and 7, the generally planar expanse (designated 54 therein) may include three white foam electrically insulative layers 56, 58, 60 of the type that are used in gel electrodes such as the medical electrode foam available from 3M®. A bottom layer 56 preferably covered or coated with what may be an electrically conductive adhesive coating or layer 70 has formed therein four electrically conductive gel electrodes (only one 42 of which is visible and only in FIG. 7) typically formed using metal powders and gels as in the formation of gel electrodes. A middle layer 58 extends around the perimeter of monitor 10 and is adhesive, thus serving when the laminar structure is conventionally cured as by heating to seal the perimeter, or edge, of the monitor. A top layer 60 is the flex circuit layer that routes signals among the circuitry components such as the battery, the electrodes and the digital or analogue processor/memory IC 13 or 13′. A conductive run of the flex circuit layer, which electrically connects electrode 42 with circuit 13, is illustrative of such circuit layer in cross-sectional view.

The flex circuit laminate or substrate for the ICs may include either a so-called complete flex or a so-called rigid flex circuit board material in which, respectively, the entirety or only a region of the patterned circuit area (shown in FIG. 7 in cross section) is flexible. It will be appreciated that—due to the very large scale integration (VLSI) of IC 13 or 13′ and the few associated circuitry 12 components including batteries 18, 18′, electrodes 20 and I/O port 28-very few signals are required to be routed in the flex circuit layer. As a result, a single-level flex circuit layer, a part of which is shown in FIG. 7 in cross section, may be formed conventionally and with very low-resolution patterning, e. g. photo-lithographic copper powder deposition, for example, thereby further reducing the cost of monitor 10.

Circuitry 12 including IC 13 or 13′ and batteries 18, 18′ may be seen essentially to be sandwiched in the void between the bottom and top layers of the foam laminate of which electrodes 20 preferably are an integral part. Preferably, IC 13 or 13′ is of the surface mount technology (SMT) type, thus producing an extremely low profile, e.g. less than approximately 0.4 cm (0.16′), laminar structure even in the central circuitry-containing region of monitor 10. Alternatively, chip-on-board techniques may be used to mount circuits and to route signals among components including ICs, batteries, electrodes and I/O ports.

Preferably, the four or more electrodes are connected to the inputs of the differential amplifier of the sensing circuit via a corresponding number of metal posts, e.g. metal post 61 electrically coupled with electrode 42, that extend outwardly from the gel electrodes and through the insulative inner layer, the posts being connected to flex circuit solder pads corresponding to such inputs, as shown. Such through connections from the inner to the outer laminar foam layer may of course be accomplished in any suitable manner, as via plated-through holes, or so-called vias, formed within a flexible, multi-layer chip-on-board circuit and interconnect configuration.

It will be appreciated that one or more outplay ports may be provided in monitor 10 to achieve a desired price-performance level and compatibility with local or remote outplay, data communication and recording equipment. Referring briefly to FIGS. 4 and 5, it may be seen that preferably one or more, e. g. four, snap connectors 62, 64, 66, 68 are provided extending from the front surface of monitor 10 for plug compatibility with 12-lead recorders. Additionally or alternatively within the spirit and scope of the invention, additional snap connectors, an RS-232 serial I/O port, an RF or IR receiver/transmitter port, a telephone jack and/or a speaker may be provided to render monitor 10 compatible with a wide variety of unidirectional or bidirectional communication, hard-copy and recording devices. It also will be appreciated that an LED or beeper may be provided that informs the patient that a recording has been made and/or that memory is full of vital sign data, so that the patient knows when to remove monitor 10 from the body and to locally outplay the data for diagnostic purposes or to surrender the monitor with its data contents intact for diagnosis at a remote site.

FIG. 5 shows monitor 10 in a second, folded configuration in which the air inlets of primary zinc-air battery 18 (not visible in FIG. S-refer to FIG. 4) is substantially closed, thereby depriving the battery of air and the circuitry of power. The controller within monitor 10 in this configuration goes into a power-save mode of operation in which memory containing a recorded vital sign graphic waveform is preserved but very little power is consumed. It will be appreciated that when monitor 10 is received at the diagnostic clinic, it very simply may be unfolded to reenergize the battery and outplayed to a desired hard-copy or recorder device such as a 12-lead recorder via the snap connectors.

In the event that the primary, preferably air-seal, e. g. zinc-air, battery 18 is dead, when monitor 10 is received at the clinic, a backup battery 18′—having a normally affixed tab 36 over its air inlet—may be used to play out the recorded cardiac waveform data. This is accomplished very simply by uncovering the air inlet over the reserve zinc-air battery. The controller, which is ‘aware’ that it has recorded ECG waveform data in its memory, preferably automatically exits the battery-save mode and-a predetermined number of seconds after the clinician unfolds the monitor-outplays the waveform data stored therein.

Broadly speaking, then, the invented disposable vital signs monitor may be seen to represent a significant improvement in portable, self-contained medical patient vital signs monitoring and control wherein such a monitor includes a generally planar expanse including a front surface and a rear surface having integral electrodes and having between the front and rear surfaces circuitry capable of sensing a vital signs signal present on the electrodes, recording the sensed signal and outplaying the recorded signal to an external device. The improvement may be understood to involve, most importantly, rendering such an expanse flexible and conformable to the shape of a patient's body, thereby to greatly improve the sensitivity and accuracy of such monitoring. Preferably, as described and illustrated herein, the monitor is also rendered self-adherent to the patient's body, thereby obviating cumbersome handling by the otherwise ambulatory patient. Also as described and illustrated herein, the monitor preferably is rendered capable of being controlled by remote telemetry, as via the provided I/O port in the wireless ones of its disclosed embodiments.

Preferably, the vital signs that are within the monitoring capability of such an improved monitor include ECG, and the monitor includes integral gel electrodes, which have been found further to increase the sensitivity and accuracy of such ECG monitoring. Within the spirit and scope of the invention, however, EEG, pulse oximetry or other continuous, real-time medical patient waveform monitoring is contemplated. In the case where ECG is the vital sign being monitored, the monitor may be rendered capable of being controlled by remote telemetry, wherein it is rendered capable of pacing a cardiac patient being monitored thereby by remote control, as described above. Moreover, as taught herein, the monitor may be rendered capable of defibrillating such a cardiac patient whose ECG is being monitored thereby.

In those cases where the vital signs being monitored include ECG, and where the monitor is equipped with cardiac event-detection capability, the monitor preferably may be equipped with a looping memory for continuous recording and window captured-data outplaying of a buffer representing—at the time of outplay thereof—a sensed ECG waveform signal that is related in time to a detected cardiac event. Such a scrolling memory feature is described in detail above and in the above-referenced patent, and, by saving on memory capacity, minimizes the circuitry required to implement the required functionality in a tiny, thin, planar flexible expanse that—due to its low cost—may be readily disposed of or recycled after use.

FIGS. 1, 4 and 5 perhaps best illustrate use of invented disposable cardiac monitor 10. FIG. 1 shows monitor 10 in its deployed configuration, albeit a lateral, cross-sectional view thereof, i.e. flattened out and adhered via a preferably conductive adhesive coating or layer 70 (shown for the sake of clarity only in FIG. 7) to a cardiac patient's chest C; FIG. 4 shows monitor 10 in its same deployed configuration but in a helpful isometric view; and FIG. 5 shows monitor 10 in its stowed configuration (in an isometric view corresponding with that of FIG. 4), i.e. bi-folded and ready to insert into a mailing envelope to send to a diagnostic center. Importantly, with monitor 10 in its deployed configuration, primary zinc-air battery 18 is operable to power circuitry 12 that senses, records and outplays vital signs waveform data, and, with monitor 10 in its stowed configuration, zinc-air battery 18 is inoperable to power circuitry 12, thus greatly extending battery life and eliminating the need for pushbuttons or other patient or physician controls.

Those of skill in the art will appreciate that the invented flexible monitor also far better conforms to the patient's chest, which may be irregular or even scarred, and utilizes gel electrodes rather than dry skin electrodes, thus increasing the integrity of cardiac waveform data sensed therethrough. Accordingly, diagnostic accuracy is improved, yet in an extremely inexpensive-to-manufacture, easy-to-use device. It also will be appreciated that the disposable vital signs monitor may find application in areas other than cardiac monitoring. For example, electroencephalograph (EEG) or pulse oximetry waveform monitoring are also possible, as well as more static medical patient vital signs monitoring such as pulse-rate, blood pressure, glucose level, blood-oxygen level, etc. Such may require a transducer of a different form to convert a patient's body characteristic signal into data suitable for recording and outplay, but any one or more lend themselves to the convenient, lightweight, inexpensive form of the invented disposable vital signs monitor.

In accordance with an alternative embodiment, a monitor that also is capable of acting as a pacer or defibrillator may be remotely controlled by a nearby transmitter to which its I/O port is programmed to respond. An ambulatory cardiac patient who is visibly experiencing tachycardia or other arrhythmia may be treated by a bystander equipped with such a portable, hand-held transmitter that may resemble, for example, a television remote control device. Valuable seconds, perhaps critical seconds, may be saved by such a remote pacer or defibrillator function provided by circuitry 12, as described above, using the proposed telemetry which requires only that I/O port 28 have bidirectional capability and that microcontroller 14 and associated circuitry provide pulse generation means, as is known.

Alternative configurations for disposable vital signs monitor 10 are contemplated as being within the spirit and scope of the invention. For example, components of the monitor may be removed from the integral flexible expanse 54, which will be referred to hereinafter as a flexible housing, to a remote, waist-worn device 72. FIG. 8 shows such a configuration in which, for example, auxiliary I/O ports 74, 76 and an auxiliary battery 78 are provided. Auxiliary I/O port 74 will be understood to support a wired or wireless, e.g. IR or RF, telecommunications and optional power interface to I/O port 28 (see FIG. 2). Auxiliary I/O port 76 will be understood to support preferably wireless, e.g. IR or RF or audible, telecommunications with, for example, a conventional telephone handset or acoustic coupler (not shown). In this configuration, power may be provided or augmented via auxiliary battery 78 to the electronics within housing 54 (via a power cable or harness not shown), as well as to the auxiliary I/O ports 74, 76.

Thus additional hardware of any suitable function may be provided in a convenient belt-worn auxiliary device 72 operatively coupled with a patient chest-worn device 10. Indeed, belt-worn device 72 may include conventional cellular telephone circuitry (including a transmitting (and perhaps also a receiving) antenna) capable at least of initiating a call to a remote patient data center and conveying vital signs data directly from chest-worn monitor 10 thereto. As shown in FIG. 8, device 72 may be suitably removably attachable to the wearer's belt with a conventional snap-on, snap-off snap, clip, slide or other suitable mechanism.

All such configurations of the invented vital signs monitor—whether integrated fully within the housing worn, for example, on the patient's chest or separately housed within an adherent patch-like housing and an external device worn on the patient's belt, arm, wrist, ankle or in a patient's purse, waist-pack, backpack or pocket—are contemplated as being within the spirit and scope of the invention. As circuit and battery miniaturization and densification continues to increase, it is contemplated that more and more functionality may be accommodated within the confines of a conveniently body-adherent, thin, laminar or monolithic integral form.

Accordingly, while the present invention has been shown and described with reference to the foregoing preferred device and method for its use, it will be apparent to those skilled in the art that other changes in form and detail may be made therein without departing from the spirit and scope of the invention as defined in the appended claims.

I claim:

1. In a portable, self contained patient vital signs monitor including a generally planar expanse including a front surface and a rear surface having integral electrodes and having between the front and rear surfaces circuitry capable of sensing a vital signs signal present on the electrodes, recording the sensed signal and outplaying the recorded signal to an external device, the improvement comprising:
said expanse being flexible, conforming to the shape of the patient's body and having a non-amorphous cross-sectional configuration.

2. The improvement of claim 1, wherein the monitor is self-adherent to the patient's body.

3. The improvement of claim 1, wherein the monitor is capable of being controlled by remote telemetry.

4. The improvement of claim 1, wherein the vital signs include ECG and wherein the monitor includes integral gel electrodes.

5. The improvement of claim 1, wherein the vital signs include ECG, wherein the monitor is capable of being controlled by remote telemetry and wherein the monitor is capable of pacing a cardiac patient being monitored thereby by remote control.

6. The improvement of claim 1, wherein the vital signs include ECG, wherein the monitor is capable of being controlled by remote telemetry and wherein the monitor is capable of defibrillating a cardiac patient being monitored thereby by remote control.

7. The improvement of claim 1, wherein the vital signs include ECG and wherein the monitor is equipped with cardiac event-detection capability and wherein the monitor is equipped with a looping memory for continuous recording and demand outplaying of a buffer representing at the time of outplay thereof a sensed ECG waveform signal that is related in time to a detected cardiac event.

8. The improvement of claim 1, wherein the monitor further includes an interface to the world-wide web.

9. The improvement of claim 1, wherein said expanse is dimensioned to conform to the size of the patient's body.

10. The improvement of claim 9 in which the sensing is of vital signs relevant to sudden infant death syndrome, wherein said expanse is dimensioned to conform to the size of a child patient's body.

11. The improvement of claim 1, wherein the monitor further includes plural external leads for monitoring plural phases of the patient's cardiography.

12. The improvement of claim 1, wherein the monitor further includes an external electronic device for telecommunicating patient vital sign data to a remote location, the external electronic device being operatively coupled with the circuitry, the external electronic device being adapted to be worn at or near the patient's waist.

13. An electrocardiograph (ECG) monitor comprising:
a flexible generally planar expanse including
a front surface;
a rear surface including a region capable of being adhered to a patient's skin, said rear surface bearing two or more electrodes;
an outplay port; and
an interior flexible circuit between said rear and front surfaces, said circuit being capable of sensing an analogue ECG signal present on said electrodes and recording in a memory that forms a part of said circuit waveform data representative of such analogue ECG signal and of producing and playing via said outplay port a signal representing said recorded waveform data for medical diagnostic purposes.

14. The monitor of claim 13, wherein said electrodes are of the gel type.

15. The monitor of claim 13, wherein said circuit includes a battery selectively operable to power and thus enable said circuit to perform such sensing, recording, said producing and said playing.

16. The monitor of claim 15, wherein said expanse is configurable in a first stowed configuration and a second deployed configuration, and wherein manipulation of said expanse from said first to said second configuration selectively operates said battery to power and thus enable said circuit.

17. The monitor of claim 16 in which said battery is of the zinc-air type having a normally exposed air inlet on the front surface of the expanse, wherein said expanse in said first configuration substantially closes said air inlet and wherein the expanse in said second configuration substantially opens said air inlet.

18. The monitor of claim 13 wherein said circuit includes an analogue-to-digital converter operatively coupling said electrodes to a digital processor operatively coupled to said memory, said processor and said memory being operatively coupled to a digital-to-analogue converter operatively coupled, in turn to said outplay port.

19. The monitor of claim 13, wherein said circuit includes an analogue signal sampling and storage device operatively coupled between said electrodes and said outplay port.

20. The monitor of claim 13, wherein said producing and said playing is of an analogue signal.

21. The monitor of claim 20, wherein said outplay port includes one or more connectors that are plug-compatible with an external n-lead ECG monitor.

22. The monitor of claim 13, wherein said producing and said playing is of a digital signal.

23. The monitor of claim 22, wherein said producing and said playing is of a digital signal suitable for conveyance on the world-wide web.

24. The monitor of claim 20, wherein said outplay port includes a serial input/output (I/O) connector.

25. The monitor of claim 13, wherein said producing and said playing is of an infrared signal.

26. The monitor of claim 13, wherein said producing and said playing is of an audio signal.

27. The monitor of claim 26, wherein said audio signal is within the radio frequency spectrum.

28. The monitor of claim 26, wherein said audio signal is suitable for trans-telephonic transmission to a remote receiver.

29. The monitor of claim 13, wherein said memory is configured as a scrolling memory and wherein said circuit is capable of recording in said scrolling memory data representative always of only the most recently sensed ECG signal.

30. The monitor of claim 29, wherein the data recording capacity of said memory is at least approximately one minute of such sensed ECG signal.

31. The monitor of claim 29, wherein the data recording capacity of said memory is at least approximately two minutes of such sensed ECG signal.

32. The monitor of claim 29, wherein the data recording capacity of said memory is at least approximately four minutes of such sensed ECG signal.

33. The monitor of claim 29, wherein said memory is an analogue signal sampling and storage device operatively coupled between said electrodes and said outplay port, and wherein the storage capacity of said device is at least approximately two minutes of such sensed ECG signal.

34. The monitor of claim 29, wherein said memory is an analogue signal sampling and storage device operatively coupled between said electrodes and said outplay port, and wherein the storage capacity of said device is at least approximately four minutes of such sensed ECG signal.

35. The monitor of claim 13, wherein said expanse is dimensioned to conform to the size of the patient's body.

36. The monitor of claim 13, wherein said circuit is further capable of sensing an analogue ECG signal indicative of sudden infant death syndrome, and wherein said expanse is dimensioned to conform to the size of a child patient's body.

37. The monitor of claim 13 in which the monitor further comprises:
plural external leads operatively coupled with the circuit for sensing and recording in the memory plural phases of the patient's cardiography.

38. The monitor of claim 13, wherein the monitor further includes an external electronic device for telecommunicating the produced and played signal to a remote location, the external electronic device being operatively coupled with the circuit, the external electronic device being adapted to be worn at or near the patient's waist.
